(19) United States
(12) Patent Application Publication
(10) Pub. No.: US 2015/0007826 A1
(43) Pub. Date: Jan. 8, 2015

(54) ENDOTRACHEAL TUBES AND SYSTEMS AND METHODS FOR EVALUATING BREATHING

(71) Applicant: H. LEE MOFFITT CANCER CENTER AND RESEARCH INSTITUTE, INC., Tampa, FL (US)

(72) Inventor: Tariq Chaudhry, Tampa, FL (US)

(21) Appl. No.: 14/372,657

(22) PCT Filed: Jan. 16, 2013
(86) PCT No.: PCT/US2013/021639

§ 371 Date: Jul. 16, 2014

Related U.S. Application Data
(60) Provisional application No. 61/586,970, filed on Jan. 16, 2012.

(51) Int. Cl. A61M 16/04

(52) U.S. Cl. CPC A61M 16/0418; A61M 16/0434; A61M 2210/1028
USPC 128/207.15; 128/207.14

(57) ABSTRACT

The present application relates to endotracheal tubes and to systems and methods for detecting airway edema and evaluating breathing with an endotracheal tube

### CROSS-REFERENCE TO RELATED APPLICATION

This application claims the benefit of U.S. Provisional Patent Application No. 61/586,970, filed on Jan. 16, 2012, entitled “Endotracheal Tubes and Systems and Methods for Evaluating Breathing,” the disclosure of which is expressly incorporated herein by reference in its entirety.

### TECHNICAL FIELD

This present application relates to endotracheal tubes and to systems and methods for evaluating laryngeal breathing and its effect on spontaneous breathing.

### BACKGROUND

During medical procedures, both surgical and non-surgical, patients must be intubated. Various medical conditions and prolonged intubations can cause swelling of the airway.

While intubated, an endotracheal tube overcomes any airway swelling and allows the free flow of the ventilator gases. During extubation, however, the presence of airway swelling can lead to airway compromise requiring urgent re-intubation.

### SUMMARY

Provided are systems and methods for evaluating breathing with a modified endotracheal tube. The details of one or more embodiments of the invention are set forth in the accompanying drawings and the description below. Other features, objects, and advantages of the invention will be apparent from the description and drawings, and from the claims.

Provided herein are endotracheal tubes. An example endotracheal tube includes an elongated tube with a distal end and a collapsible portion located along the tube length. Furthermore, when the endotracheal tube is inserted in a subject, the location of the collapsible portion is positionable in the vocal cord region of the subject.

Optionally, the endotracheal tube includes an inflatable cuff. The inflatable cuff is optionally positioned about the endotracheal tube between the distal end and the collapsible portion.

Optionally, the collapsible portion of the endotracheal tube is located a predetermined distance from the distal end of the endotracheal tube or from the cuff. The cuff optionally has a proximal edge. Optionally, the collapsible portion is located a predetermined distance from the proximal edge of the cuff.

The collapsible portion is optionally configured to contact the vocal cords of the subject. Optionally, the collapsible portion is moveable between a non-collapsed and collapsed configuration. For example, movement of the vocal cords causes movement of the collapsible portion between the non-collapsed and collapsed configuration. Optionally, the collapsed configuration allows airflow caused by respiration of the subject to pass between the endotracheal tube and the vocal cords of the subject. Optionally, the collapsible portion is biased towards its collapsed configuration.

Optionally, the collapsible portion is moveable between its collapsed and non-collapsed configuration. For example, the collapsible portion may be naturally biased towards its collapsed configuration. Optionally, the collapsible portion is expandable to its non-collapsible configuration by inserting a stent device into the endotracheal tube lumen.

Optionally, gas is delivered to the subject through the endotracheal tube and/or the stent device. Optionally, the collapsible portion of the endotracheal tube comprises an elastic membrane. The collapsible portion optionally has a distal edge. Optionally, the distal edge of the collapsible portion is spaced from the proximal edge of the cuff by a distance of about 1 inch or more. Optionally, the length of the collapsible portion is between about 1 inch and about 2 inches or more. The predetermined length may optionally be the entire length of the outer tube.

For example, the collapsible portion optionally comprises the full circumference of the tube over a predetermined length of the tube. The predetermined length can be between about 1 inch and about 2 inches. The predetermined length may optionally be the entire length of the outer tube. Optionally, the endotracheal tube distal and proximal to the collapsible portion comprises standard endotracheal tube structure. For example, the standard endotracheal tube structure is not collapsible under normal clinical conditions.

Also provided is an endotracheal access system. The endotracheal access system includes an endotracheal tube, which has a distal end and a collapsible portion located at a position along the tube length. Furthermore, the collapsible portion of the endotracheal tube allows for positioning of the collapsible portion in the vocal cord region of a subject when the endotracheal tube is inserted in the subject. The endotracheal access system further includes a stent configured for positioning within the lumen of the endotracheal tube. The stent holds the collapsible portion of the endotracheal tube in a non-collapsed configuration.

Optionally, the stent is slidably moveable within the endotracheal tube lumen. The collapsible portion is optionally biased to collapse when not held in the non-collapsed configuration by the stent. Optionally, the collapsible portion moves between its non-collapsed configuration and its collapsed configuration upon withdrawal of the stent from within the lumen defined by the collapsible portion. For example, the stent may be advanced back through the lumen of the collapsed configuration of the collapsible portion. This advancement of the stent optionally causes the collapsible portion to regain its non-collapsed configuration.

Optionally, the stent is advanceable and/or retractable from a position external to the oral cavity of the subject. Optionally, an inflatable cuff may be positioned about the endotracheal tube between the distal end of the endotracheal tube and the collapsible portion. The collapsible portion is optionally located a predetermined distance from the distal end of the endotracheal tube or from the cuff. For example, the cuff has a proximal edge, and the collapsible portion is optionally located a predetermined distance from the proximal edge of the cuff.

The collapsible portion is optionally configured to contact the vocal cords of the subject. For example, movement of the vocal cords causes movement of the collapsible portion between the non-collapsed and collapsed configuration. The collapsed configuration optionally allows airflow caused by respiration of the subject to pass between the endotracheal tube and the vocal cords of the subject.

Optionally, gas is deliverable to the subject through the endotracheal tube and/or stent device. The collapsible portion optionally comprises an elastic membrane. Optionally, the collapsible portion comprises a distal edge. The distal edge of the collapsible portion, for example, is spaced from the proximal edge of the cuff by a distance of about 1 inch or more. Optionally, the length of the collapsible portion is between about 1 inch and about 2 inches or more. The collapsible portion may optionally be the entire length of the outer tube.

The collapsible portion optionally comprises the full circumference of the tube over a predetermined length of the tube. The endotracheal tube distal and proximal to the collapsible portion optionally comprises standard endotracheal tube structure. The standard endotracheal tube structure is optionally not collapsible under normal clinical conditions. Optionally, the subject has airway edema, prolonged intubation, vocal cord injury, obesity, prolonged head down procedures, difficult airway, or any pediatric procedure requiring an intubation.

Also provided is a method for evaluating breathing in a subject. This method includes positioning an endotracheal tube in the subject. The endotracheal tube has a distal end and a collapsible portion located at a position along the tube length. The location of the collapsible portion is in the vocal cord region of the subject. The method also includes positioning a stent in the endotracheal tube. The stent holds the collapsible portion in a non-collapsed configuration. The method further includes removing the stent such that the collapsible portion assumes a collapsed configuration. The method also includes characterizing the breathing of the subject around the collapsed configuration of the collapsible portion to evaluate breathing of the subject.

For example, the evaluation of the breathing optionally indicates that the subject is able to breathe spontaneously. Optionally, the method includes the removal of the endotracheal tube from the subject.

In other examples, the evaluation of the breathing optionally indicates that the subject is not able to breathe spontaneously or has clinically reduced capacity to breathe without intubation-aided assistance. The method optionally includes repositioning the stent in the collapsible portion to cause it to assume its non-collapsed configuration. Optionally, the method includes the repeated removal of the stent such that the collapsible portion assumes a collapsed configuration. The breathing of the subject around the collapsed configuration of the collapsible portion is characterized to evaluate the breathing of the subject.

The subject on which the methods, devices, and systems are used optionally has airway edema, prolonged intubation, vocal cord injury, obesity, prolonged head down procedures, difficult airway, or any pediatric procedure requiring an intubation. Optionally, the method includes the evaluation of breathing of a subject to determine that the subject has airway edema or vocal cord injury.

### DESCRIPTION OF DRAWINGS

FIG. 1A is a schematic illustration of an example endotracheal tube with a collapsible portion.

FIG. 1B is a schematic illustration of an example stent.

FIG. 2 is a schematic illustration of an example endotracheal tube with a collapsible portion.

FIG. 3A is a schematic illustration of an example system for evaluating breathing with an endotracheal tube having a collapsible portion.

FIG. 3B is a schematic illustration of an example system for evaluating breathing with an endotracheal tube having a collapsible portion.

FIG. 4A is a schematic illustration of a cross section through the vocal cords of a subject with an endotracheal tube supporting the vocal cords.

FIG. 4B is a schematic illustration of a cross section through the vocals cords of a subject with an endotracheal tube having a collapsed portion positioned between the vocal cords.

FIG. 4C is a schematic illustration of a cross section through the vocals cords of a subject with an endotracheal tube having a collapsed portion positioned between the vocal cords.

FIG. 5A is a schematic illustration of a cross section through the vocals cords of a subject with an endotracheal tube having a collapsed portion positioned between the vocal cords.

FIG. 5B is a schematic illustration of a cross section through the vocal cords of a subject with an endotracheal tube supporting the vocal cords.

FIG. 5C is a schematic illustration of a cross section through the vocal cords of a subject with an endotracheal tube having a collapsed portion positioned between the swollen vocal cords.

Like reference symbols in the various drawings indicate like elements.

### DETAILED DESCRIPTION

The present invention now will be described more fully hereinafter with reference to specific embodiments of the invention. Indeed, the invention can be embodied in many different forms and should not be construed as limited to the embodiments set forth herein; rather, these embodiments are provided so that this disclosure will satisfy applicable legal requirements.

Patients are intubated when undergoing an array of medical procedures, including both surgical and non-surgical procedures. Under prolonged intubations, a patient may experience airway edema and/or swelling. Furthermore, certain medical conditions, such as obesity, can cause airway edema. While intubated, a commonly-used endotracheal tube overcomes any airway swelling that may be present to allow the free-flow of the ventilator gases.

In the presence of airway swelling, extubation is risky and can lead to airway compromise requiring urgent re-intubation. Aside from clinical judgment by a medical professional, no test accurately predicts airway swelling. In order for a medical professional to make a clinical judgment regarding airway swelling, an intubated patient must be fully awake. Thus, sedatives at this point may not be used because the patient needs to be cooperative and able to follow commands The high level of anxiety in awake and intubated patients can lead to hypertension and tachycardia, potentially complicating the existing medical conditions.

In the event that a patient is prematurely extubated, the clinical and professional management of this premature extubation adds to the medical expenses in the form of medications, medical supplies, equipment, and personnel. On the other hand, a more conservative extubation approach leads to an unnecessarily prolonged intubation, which also adds to the medical expenses in acute-care facilities.

Provided herein are systems and methods for detecting airway edema and evaluating breathing with a modified endotracheal tube. Such systems and methods can be used, for example, to intubate patients and evaluate breathing during the extubation process. For example, provided is an endotracheal tube used to assess airway edema and swelling in intubated patients. This endotracheal tube can help medical professionals assess airway flow in intubated patients.

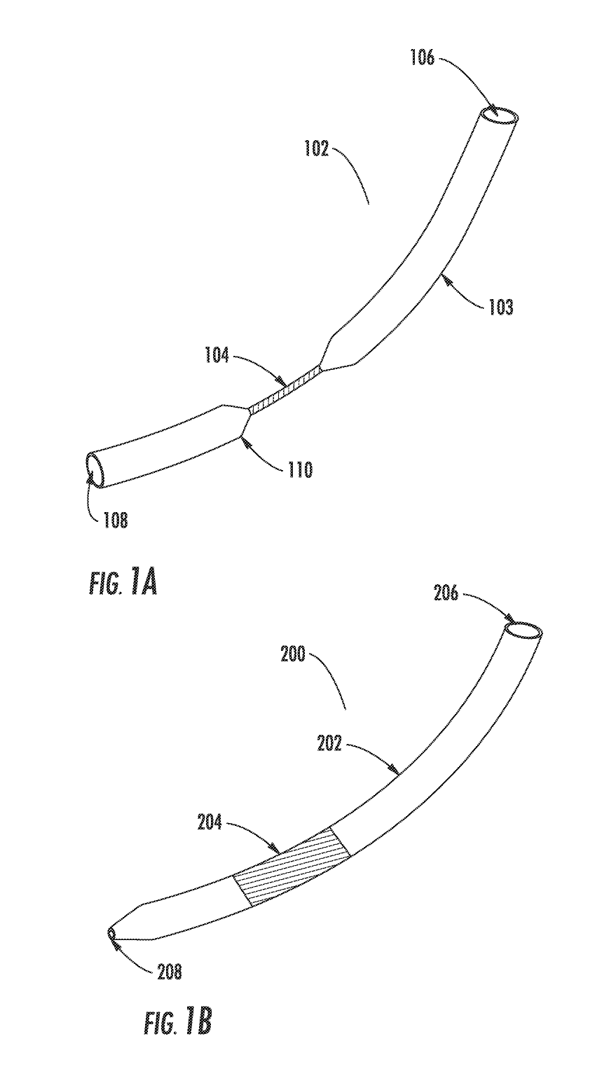

Referring to FIG. 1A, an example endotracheal tube is shown. Endotracheal tube 102 can be used in an intubated patient to assess airway flow. The endotracheal tube 102 includes an elongated tube 103 with a distal end 108 and a collapsible portion 104 located along the tube length. Furthermore, when the endotracheal tube 102 is inserted in a subject, the location of the collapsible portion 104 allows for positioning of the collapsible portion 104 in the vocal cord region of the subject.

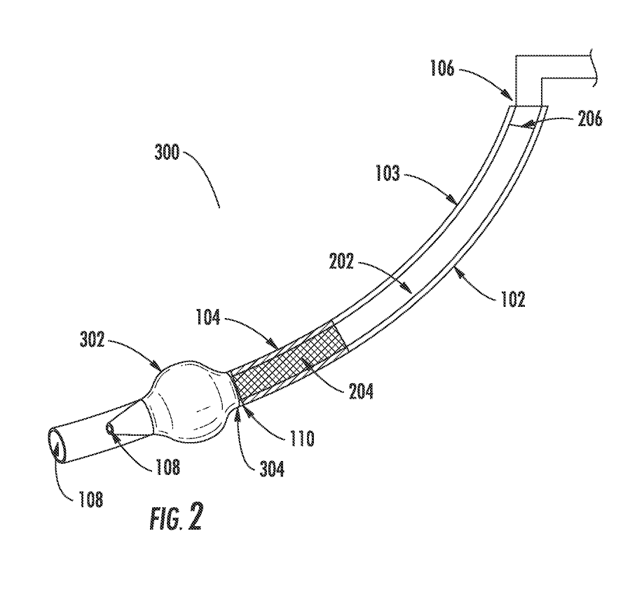

An example endotracheal tube 300 can also include a cuff 302 as shown in FIG. 2. Endotracheal tube 300 optionally includes an inflatable cuff 302. The inflatable cuff 302 is optionally positioned about the endotracheal tube between the distal end 108 and the collapsible portion 104. The cuff itself may optionally be inflated or deflated by an operator. Under normal clinical conditions, the cuff is deflated during the intubation procedure of a patient, so that an endotracheal tube may be inserted into the patient. Once the endotracheal tube is correctly positioned in the patient, the operator of the cuff may inflate the cuff. Once inflated, the cuff impedes flow of gases into or out of the patient. Instead, a mechanical ventilator distributes gases to and from the patient through an endotracheal tube. Prior to extubation, the cuff is deflated. After deflation of the cuff, the cuff and endotracheal tube may be removed from the patient.

Optionally, endotracheal tube 102 or 300 includes a collapsible portion 104. The collapsible portion 104 is optionally located a predetermined distance from the distal end 108 of the endotracheal tube 102 or 300 or from the cuff 302. The cuff 302 optionally has a proximal edge 304. Optionally, the collapsible portion 104 is located a predetermined distance from the proximal edge 304 of the cuff 302. The collapsible portion 104 is optionally configured to contact the vocal cords of the subject.

Within the airway, the vocal cords comprise the narrowest opening. The collapsible portion resides at a predetermined location on the endotracheal tube. For example, about 3 inches away from the distal end of the endotracheal tube or about 1 inch from the proximal edge of the cuff. The part of the endotracheal tube that remains outside of the subject is optionally marked to indicate to the operator or medical professional how much of the endotracheal tube has been inserted into the subject. Therefore, the medical professional can assess when the collapsible portion of the endotracheal tube is positioned at the vocal cords. The operator or medical professional has flexibility to move the endotracheal tube into or out of the subject, so that the collapsible portion seats between the vocal cords. The collapsible portion can be contacting the vocal cords, but also some portions of the collapsible portion may be outside the vocal cord region.

The collapsible portion 104 has a distal edge 110. Optionally, the distal edge 110 of the collapsible portion 104 is spaced from the proximal edge 304 of the cuff 302 by a distance of about 1 inch or more.

Optionally, the collapsible portion 104 of the endotracheal tube 102 or 300 comprises an elastic membrane 204. The elastic membrane 204 of the collapsible portion 104 optionally has properties such that the natural or normal position for the collapsible portion is in a collapsed configuration. For example, unless supported by a stent, or some other support device or system, the elastic membrane 204 optionally remains in a collapsed configuration. The elastic membrane 204 portion is optionally made from a flexible collapsible rubber or polyvinyl chloride (PVC). The other portions of the endotracheal tube are optionally made of PVC.

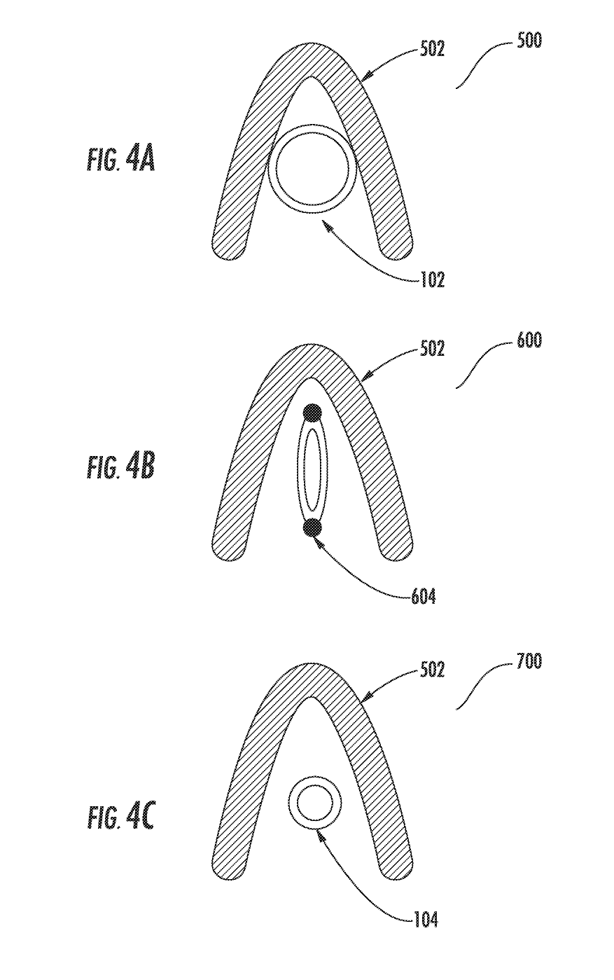

FIGS. 4A, 4B, and 4C are schematic illustrations showing endotracheal tubes positioned in between subject's vocal cords. For example, referring to FIG. 4A, an example endotracheal tube 102 is shown supporting the vocal cords in a non-collapsed configuration 500. Referring now to FIG. 4B, an example endotracheal tube 604 is shown in a collapsed configuration 600. Endotracheal tube 604 includes pillars on two opposite sides of the endotracheal tube to add support to the collapsible portion of the endotracheal tube. The two supporting pillars are connected with an elastic membrane 204. Referring to FIG. 4C, an example endotracheal tube with collapsible portion 104 is shown in a collapsed configuration 700. In FIG. 4C, the entire portion of the endotracheal tube in the vocal cord region is made of an elastic membrane 204, whereas FIG. 4B shows an endotracheal tube with two supporting pillars, which optionally comprise a PVC material.

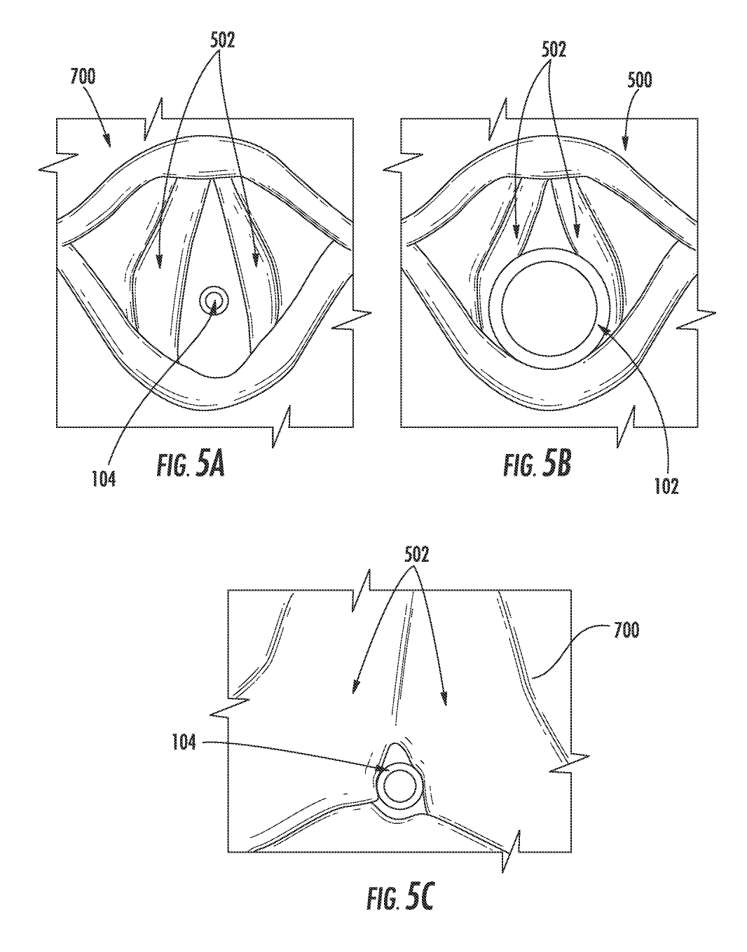

FIGS. 5A, 5B, and 5C are schematic illustrations showing endotracheal tubes positioned in between subject's vocal cords. For example, referring to FIG. 5A, an example endotracheal tube with collapsible portion 104 is shown in a collapsed configuration 700. Referring to FIG. 5B, another example endotracheal tube 102 is shown supporting the vocal cords in a non-collapsed configuration 500. Referring now to FIG. 5C, an example endotracheal tube with collapsible portion 104 is shown in a collapsed configuration 700. FIG. 5C represents the cross section of the vocal cords after the stent is retracted through collapsible portion 104. FIG. 5C also shows that the airway is swollen and there is little to no room around the collapsed tube for subject to breathe. Under the conditions shown in FIG. 5C, the subject may show signs of respiratory distress, stridor, or no breathing at all. When signs of respiratory distress are present, extubation is not safe. The stent 202 is advanced back through the collapsible portion 104, so that the collapsible portion 104 re-expands to non-collapsed configuration 500. The subject remains on mechanical ventilation until the swelling resolves and extubation is safe.

Optionally, the collapsible portion 104 is moveable between a non-collapsed 500 and collapsed 700 configuration. For example, movement of the vocal cords optionally causes movement of the collapsible portion 104 between the non-collapsed 500 and collapsed 700 configuration. Optionally, the collapsed configuration 700 allows airflow caused by respiration of the subject to pass between endotracheal tube 102 and the vocal cords of the subject. Optionally, the collapsible portion 104 is biased towards its collapsed configuration 700.

The bias toward the collapsed configuration optionally results from the contact of the collapsible portion with the vocal cords. The movement or force experienced from the vocal cords optionally causes the collapsible portion to assume a collapsed configuration. In other examples, the elasticity and physical makeup and/or other characteristics of the collapsible portion optionally urge the collapsible portion to move to a collapsed configuration under normal or natural conditions.

Referring now to FIG. 1B, an example stent device 200 is shown. For example, the stent can be an elongated tubal device. The stent device optionally comprises a PVC-type material or a metal. The outer circumference of the tubal stent is optionally less than the inner circumference of the endotracheal tube, such that the stent can fit inside the endotracheal tube lumen. Optionally, the ratio of the circumferences are such that the stent can slide freely within the endotracheal tube with an appropriate amount of friction. For example, an operator can advance or withdraw the stent in the lumen.

Optionally, the collapsible portion 104 is expandable to its non-collapsible configuration 500 by inserting a stent device 202 into the endotracheal tube 102 lumen. Optionally, gas is delivered to the subject through the endotracheal tube 102 or 300 and/or the stent device 202.

Optionally, the length of the collapsible portion 104 is between about 1 inch and about 2 inches or more. The length of the collapsible portion 104 may also extend the entire length of the tube 102. For example, the collapsible portion 104 comprises the full circumference of the tube over a predetermined length of the tube. The elastic membrane 204 optionally comprises a flexible collapsible rubber or PVC. Under normal or natural conditions, the elastic membrane 204 is biased, or remains, in a collapsed position. The elastic membrane 204 optionally spans along the endotracheal tube for between about 1 inch and about 2 inches or more. The elastic membrane 204 optionally contacts the vocal cords. When a stent, or another support, is placed in the elastic membrane 204 lumen, the elastic membrane 204 expands around the circumference of the stent or support. Optionally, the endotracheal tube distal and proximal to the collapsible portion 104 comprises standard endotracheal tube structure. For example, the standard endotracheal tube structure is not collapsible under normal clinical conditions. The standard endotracheal tube is optionally made of PVC.

During intubation, swelling of the airway and the vocal cords makes an extubation assessment challenging. Using a current endotracheal tube, when an endotracheal tube is pulled out of a patient, and the patient is unable to maintain the airway, the patient must be re-intubated. Multiple instrumentation of the airway is extremely uncomfortable for a patient who is awake. The agitation for the patient can result in hypertension and tachycardia for the patient, as well as trauma and bleeding in the airway.

Also provided in the present application is an endotracheal access system. This system allows for minimal stimulation and discomfort for the awake patient, as well as stable hemodynamics. Furthermore, extubation is timelier, with reduced airway trauma and little to no product or resource waste.

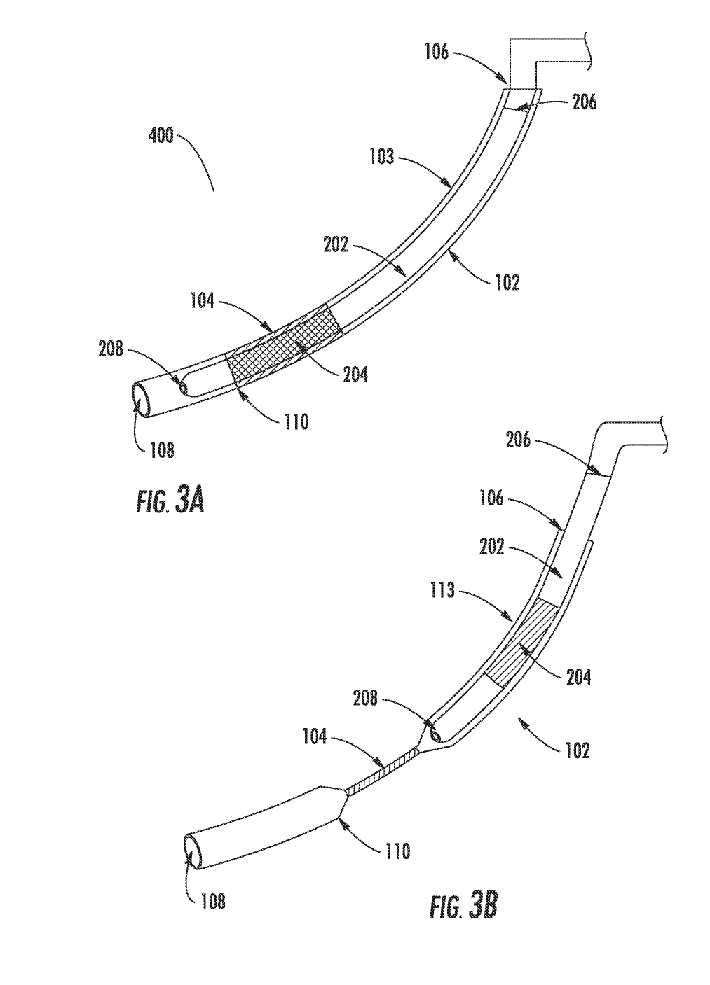

Referring to FIG. 3A and FIG. 3B, an example endotracheal access system 400 is shown. The endotracheal access system 400 includes an endotracheal tube 102, which has a distal end 108 and a collapsible portion 104 located at a position along the tube length. Furthermore, the collapsible portion 104 of the endotracheal tube 102 allows for positioning of the collapsible portion 104 in the vocal cord region of a subject when the endotracheal tube 102 is inserted in the subject. The endotracheal access system 400 further includes a stent 202 configured for positioning within the lumen of the endotracheal tube 102. The stent 202 holds the collapsible portion 104 of the endotracheal tube 102 in a non-collapsed configuration 500.

Optionally, the stent 202 is slidably moveable within the endotracheal tube 102 lumen. For example, the collapsible portion 104 is biased to collapse when not held in the non-collapsed configuration 500 by the stent 202. Optionally, the collapsible portion 104 moves between its non-collapsed configuration 500 and its collapsed configuration 700 upon withdrawal of the stent 202 from within the lumen defined by the collapsible portion 104. For example, the stent 202 may be advanced back through the lumen of the collapsed configuration 700 of the collapsible portion 104. This advancement of the stent 202 causes the collapsible portion 104 to regain its non-collapsed configuration 500.

Optionally, the stent 202 is advanceable and/or retractable from a position external to the oral cavity of the subject. When the patient is intubated, intubation progresses as usual with the stent inserted in the endotracheal tube lumen. The endotracheal tube and stent are inserted into the patient, and the cuff is inflated to prevent air leakage around the tube. Referring to FIG. 3A, an example endotracheal access system is shown with the stent 202 in place and the collapsible portion 104 in a non-collapsed configuration 500. Once the relevant medical procedure is complete, the patient is evaluated for extubation. At this point, the stent 202 may optionally be retracted through the endotracheal tube, and this movement is accomplished through the stent sliding through the endotracheal tube lumen. Referring now to FIG. 3B, an example endotracheal access system is shown with the stent 202 in a retracted position. Once the stent is retracted, the collapsible portion 104 optionally assumes its collapsed configuration 700. In the event that extubation is not the appropriate medical decision, then the stent may be advanced back through the endotracheal into the position as shown in FIG. 3A. The advancement or retraction of the stent is performed by a medical professional, for example, on a portion of the tube that is external to the patient and the patient's oral cavity.

Optionally, an inflatable cuff 302 may be positioned about the endotracheal tube 102 or 300 between the distal end 108 of the endotracheal tube 102 or 300 and the collapsible portion 104. The collapsible portion 104 is optionally located a predetermined distance from the distal end 108 of the endotracheal tube 102 or 300 or from the cuff 302. For example, the cuff 302 has a proximal edge 304, and the collapsible portion 104 is optionally located a predetermined distance from the proximal edge 304 of the cuff 302.

During intubation, the cuff is inflated to prevent air leakage. A mechanical ventilator optionally delivers gases to and from the subject. Prior to the extubation process, the cuff is optionally deflated. The stent 202 is optionally retracted, which allows the collapsible portion of the endotracheal tube to assume a collapsed configuration. The medical professional may then request that the subject breathe or attempt to breathe. Because the cuff is deflated, air flows around the cuff. The endotracheal tube is also in a collapsed configuration, which allows airflow through the vocal cords, unless swelling or some other medical condition inhibits the airflow, for example as shown in FIG. 5C.

The collapsible portion 104 is optionally configured to contact the vocal cords of the subject. For example, movement of the vocal cords causes movement of the collapsible portion 104 between the non-collapsed 500 and collapsed 700 configuration. The collapsed configuration 700 optionally allows airflow caused by respiration of the subject to pass between the endotracheal tube 102 or 300 and the vocal cords of the subject.

Optionally, gas is deliverable to the subject through the endotracheal tube 102 or 300 and/or stent device 202. The collapsible portion 104 optionally comprises an elastic membrane 204. Optionally, the collapsible portion comprises a distal edge 110. The distal edge 110 of the collapsible portion 104, for example, is spaced from the proximal edge 304 of the cuff 302 by a distance of about 1 inch or more. Optionally, the length of the collapsible portion 104 is between about 1 inch and about 2 inches or more.

The collapsible portion 104 optionally comprises the full circumference of the tube 102 or 300 over a predetermined length of the tube 102 or 300. For example, the endotracheal tube 102 or 300 distal and proximal to the collapsible portion 104 comprises standard endotracheal tube structure. The standard endotracheal tube structure is optionally not collapsible under normal clinical conditions. Optionally, the subject has airway edema, prolonged intubation, vocal cord injury, obesity, prolonged head down procedures, difficult airway, or any pediatric procedure requiring an intubation.

Also provided in the present application is a method for evaluating breathing in a subject. Under this method, a patient is intubated without any modified intubation techniques, and there are no modifications necessary for the ventilator settings or ventilator management. During extubation and with the described tubes and systems, the patient's ability to comfortably breathe reflects an airway that is suitable for a safe extubation. The patient is able to communicate with medical professionals prior to extubation. In the event that swelling of the airway is present, breathing characteristics, such as stridor, signals to the medical professionals present that extubation should not proceed. The patient should thus be placed back on mechanical ventilation. In extreme cases, no breathing occurs after a patient is extubated and must be placed back on mechanical ventilation.

This method for evaluating the breathing of a subject includes positioning an endotracheal tube 102 or 300 in the subject. The endotracheal tube 102 or 300 has a distal end 108 and a collapsible portion 104 located at a position along the tube length. The location of the collapsible portion 104 is in the vocal cord region of the subject. The method also includes positioning a stent 202 in the endotracheal tube 102 or 300. The stent 202 holds the collapsible portion 104 in a non-collapsed configuration 500. The method further includes removing the stent 202 such that the collapsible portion 104 assumes a collapsed configuration 700. The method also includes characterizing the breathing of the subject around the collapsed configuration 700 of the collapsible portion 104 to evaluate breathing of the subject. Characterizing the breathing optionally includes determining whether the breathing is spontaneous or not. Characterizing may also include determining the presence, absence, or extent of labored breathing, stridor, abnormal airway sounds, abnormal muscular tension, or other breathing characteristics that indicate whether the patient should be extubated.

During extubation, the cuff 302 is deflated, and the stent 202 is optionally retracted through the endotracheal tube to allow the collapsible section 104 to assume a collapsed configuration 700. In the collapsed configuration, airflow optionally flows around the collapsible portion 104. In the event that swelling or airway edema does not exist, then the subject should breathe spontaneously. For example, the evaluation of the breathing indicates that the subject is able to breathe spontaneously. Optionally, the method includes the removal of the endotracheal tube 102 from the subject if characterization suggests the ability to breathe spontaneously. The ability of the subject to breathe spontaneously suggests to medical professionals that extubation may be safely performed.

The evaluation of the breathing optionally indicates that the subject is not able to breathe spontaneously or has clinically reduced capacity to breathe without intubation-aided assistance. In the event that the subject is not able to breathe spontaneously, then the stent 202 may be advanced back through the endotracheal tube to optionally allow for mechanical ventilation. The lack of ability of the subject to breathe spontaneously or the presence of other pathological breathing characteristics indicates that even though the collapsible portion is in its collapsed configuration, the subject cannot breathe spontaneously around the collapsible portion, or cannot breath appropriately or safely without artificial ventilation.

The method optionally includes repositioning the stent 202 in the collapsible portion 104 to cause it to assume its non-collapsed configuration 500, for example when there is a reduced breathing capacity. Optionally, the method includes the repeated removal of the stent 202 such that the collapsible portion 104 assumes a collapsed configuration 700. The breathing of the subject around the collapsed configuration 700 of the collapsible portion 104 is characterized to evaluate the breathing of the subject. For example, the subject has airway edema, prolonged intubation, vocal cord injury, obesity, prolonged head down procedures, difficult airway, or any pediatric procedure requiring an intubation. Optionally, the method includes the evaluation of breathing of a subject to determine that the subject has airway edema or vocal cord injury.

A number of embodiments of the invention have been described. Nevertheless, it will be understood that various modifications may be made without departing from the spirit and scope of the invention. Accordingly, other embodiments are within the scope of the following claims.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the materials for which they are cited are hereby specifically incorporated by reference in their entireties.

1. An endotracheal tube, comprising an elongated tube having a distal end and a collapsible portion located at a position along the tube length, the location of the collapsible portion allowing for positioning of said collapsible portion in the vocal cord region of a subject when the endotracheal tube is inserted in the subject.

2. The endotracheal tube of claim 1, wherein an inflatable cuff is positioned about the endotracheal tube between the distal end and the collapsible portion.

3. The endotracheal tube of claim 1, wherein the collapsible portion is located a predetermined distance from the distal end of the endotracheal tube or from the cuff.

4. The endotracheal tube of claim 3, wherein the cuff has a proximal edge and where the collapsible portion is located a predetermined distance from the proximal edge of the cuff.

5. The endotracheal tube of claim 1, wherein the collapsible portion is configured to contact the vocal cords of the subject.

6. The endotracheal tube of claim 5, wherein the collapsible portion is moveable between a non-collapsed and collapsed configuration.

7. The endotracheal tube of claim 6, wherein movement of the vocal cords causes movement of the collapsible portion between the non-collapsed and collapsed configuration.

8. The endotracheal tube of claim 6, wherein the collapsed configuration allows airflow caused by respiration of the subject to pass between the endotracheal tube and the vocal cords of the subject.

9. The endotracheal tube of claim 6, wherein the collapsible portion is biased towards its collapsed configuration.

10. The endotracheal tube of claim 9, wherein the collapsible portion is moveable between its collapsed and non-collapsed configuration.

11.-19. (canceled)

20. An endotracheal access system, comprising:
a. an endotracheal tube having a distal end and a collapsible portion located at a position along the tube length, the location of the collapsible portion allowing for positioning of said collapsible portion in the vocal cord region of a subject when the endotracheal tube is inserted in the subject; and
b. a stent configured for positioning within the lumen of the endotracheal tube, the stent holding the collapsible portion in a non-collapsed configuration.

21. The system of claim 20, wherein the stent is slidably moveable within the endotracheal tube lumen.

22. The system of claim 20, wherein the collapsible portion is biased to collapse when not held in the non-collapsed configuration by the stent.

23. The system of claim 22, wherein the collapsible portion moves between its non-collapsed configuration and its collapsed configuration upon withdrawal of the stent from within the lumen defined by the collapsible portion.

24. The system of claim 23, wherein advancing the stent back through the lumen of the collapsed configuration of the collapsible portion causes the collapsible portion to regain its non-collapsed configuration.

25. The system of claim 24, wherein the stent is advanceable and or retractable from a position external to the oral cavity of the subject.

26. The system of claim 20, wherein an inflatable cuff is positioned about the endotracheal tube between the distal end of the endotracheal tube and the collapsible portion.

27. The system of claim 20, wherein the collapsible portion is located a predetermined distance from the distal end of the endotracheal tube or from the cuff.

28. The system of claim 26, wherein the cuff has a proximal edge and where the collapsible portion is located a predetermined distance from the proximal edge of the cuff.

29. The system of claim 20, wherein the collapsible portion is configured to contact the vocal cords of the subject.

30.-48. (canceled)
